(19) United States
(12) Patent Application Publication
(10) Pub. No.: US 2024/0384357 A1
(43) Pub. Date: Nov. 21, 2024

(54) NUCLEIC ACID IMPLEMENTATION OF MULTILAYER PERCEPTRONS

(71) Applicant: The Regents of the University of California, Oakland, CA (US)

(72) Inventor: Roy WOLLMAN, Los Angeles, CA (US)

(73) Assignee: The Regents of the University of California, Oakland, CA (US)

(21) Appl. No.: 18/696,746

(22) PCT Filed: Oct. 3, 2022
(86) PCT No.: PCT/US22/45541

§ 371 Date: Mar. 28, 2024

Related U.S. Application Data
(60) Provisional application No. 63/251,963, filed on Oct. 4, 2021.

(51) Int. Cl. C12Q 1/6886

(52) U.S. Cl. CPC C12Q 1/6886; C12Q 2600/158

(57) ABSTRACT

Methods for utilizing a nucleic acid-based multilevel perceptron are provided.

### CROSS-REFERENCE TO RELATED APPLICATIONS

This application claims priority to U.S. Provisional Patent Application Ser. No. 63/251,963, filed Oct. 4, 2021, which is incorporated herein by reference in its entirety.

### TECHNICAL FIELD

The invention is in the field of DNA computing.

### BACKGROUND

A DNA computer is a design that leverages the properties of DNA molecules to solve computational problems. There are several implementations of how these computations are done. Most are based on a series of hybridization of complementary base-pairs of nucleic acids. After the series of hybridization, the final output of the computation is read, either by sequencing or through final hybridization with nucleic acid oligomers (oligos) that are conjugated to fluorophores. See, for example, Qian, Lulu; Winfree, Erik; Bruck, Jehoshua (July 2011). “Neural network computation with DNA strand displacement cascades”. Nature. 475 (7356): 368-372; and Cherry, Kevin M.; Qian, Lulu (2018 Jul. 4). “Scaling up molecular pattern recognition with DNA-based winner-take-all neural networks”. Nature. 559 (7714): 370-376.

New methods for utilizing nucleic acids for computational analysis using a multilayer perceptron, a type of neural network, to help solve problems of regression and classification, among other purposes are needed.

### SUMMARY

In one embodiment, a method is provided for identifying at least one preselected relationship among a plurality of nucleic acid molecule species in a sample, the method comprising the steps of:
a. binding the plurality of nucleic acid molecule species to a substrate;
b. incubating the substrate with a plurality of first weight oligonucleotides, wherein the detector sequences of the first weight oligonucleotides bind to any nucleic acid molecule species complementary thereto bound to the substrate;
c. removing unbound first weight oligonucleotides;
d. incubating the substrate with first extender oligonucleotides and optionally first blocking oligonucleotides, wherein the detector sequences on the first extender oligonucleotides or the first blocking oligonucleotides, if used, bind to sites complementary thereto on the bridging sequences of the first weight oligonucleotides, the extent of the binding of the first extender nucleotides based on the predetermined affinity of the first blocking oligonucleotides, if used, and on the detector sequences on the first extender oligonucleotides to the bridging sequences of the first weight oligonucleotides;
e. removing unbound first extender oligonucleotides and first blocking oligonucleotides if used;
f. wherein the bound first extender oligonucleotides are final extender oligonucleotides, or wherein optionally one or more repeats of steps b through e are conducted with further weight oligonucleotides, optional further blocking oligonucleotides and further extender oligonucleotides, to provide additional determination of the preselected relationship, and wherein the last repeat of steps b through e provide final extender oligonucleotides; and
g. incubating the substrate with a plurality of readout oligonucleotides, each readout oligonucleotide comprising a detectable label bound to an oligonucleotide complementary to the bridging sequence on the final extender oligonucleotide, thereby detecting the extent of bound final extender oligonucleotides on the substrate; wherein
   i. each first weight oligonucleotide comprises two segments, one segment comprising a detector sequence complementary to a nucleic acid molecule species or sequence therein of interest, and the other segment comprising a bridging sequence complementary to a first blocking oligonucleotide or to the detector sequence of a first extender oligonucleotide;
   ii. each first blocking oligonucleotide comprises a predetermined affinity to the bridging sequence of the first weight oligonucleotide; and
   iii. each first extender oligonucleotide comprises two segments, a detector sequence complementary to the bridging sequence of the first weight oligonucleotide, and a bridging sequence complementary to the detector sequence on a further weight oligonucleotide or readout oligonucleotide; and wherein the extent of binding of the readout oligonucleotides to the substrate provides the at least one preselected relationship among the plurality of nucleic acid molecule species in the sample.

In one embodiment, a method is provided for identifying at least one preselected relationship among a plurality of nucleic acid molecule species in a sample, the method comprising the steps of:
a. binding the plurality of nucleic acid molecule species to a substrate;
b. incubating the substrate with a plurality of first weight oligonucleotides, wherein the detector sequences of the first weight oligonucleotides bind to any nucleic acid molecule species complementary thereto bound to the substrate;
c. removing unbound first weight oligonucleotides;
d. incubating the substrate with first extender oligonucleotides and optionally first blocking oligonucleotides, wherein detector sequences of the first extender oligonucleotides and the first blocking oligonucleotides, if used, bind to sites complementary thereto on the bridging sequences of the first weight oligonucleotides, the extent of the binding of the extender nucleotides based on the predetermined affinity of the first blocking oligonucleotides, if used, and on the first extender nucleotides to the bridging sequences of the first weight oligonucleotides;
e. removing unbound first extender oligonucleotides and first blocking oligonucleotides if used;
f. incubating the substrate with a plurality of second weight oligonucleotides, wherein the detector sequences of the second weight oligonucleotide bind to complementary bridging sequences on the first extender oligonucleotides;
g. removing unbound second weight oligonucleotides;
h. incubating the substrate with second extender oligonucleotides and optionally second blocking oligonucleotides, wherein detector sequences of the second extender oligonucleotides and the second blocking oligonucleotides, if used, bind to sites complementary thereto on the bridging sequences of the second weight oligonucleotides, the extent of the binding of the second extender nucleotides based on the predetermined affinity of the second blocking oligonucleotides, if used, and on the second extender nucleotides to the bridging sequences of the second weight oligonucleotides;
i. removing unbound second extender oligonucleotides and second blocking oligonucleotides if used;
j, wherein the bound second extender oligonucleotides are final extender oligonucleotides, or wherein optionally one or more repeats of steps f through i are conducted with further weight oligonucleotides, further blocking oligonucleotides and further extender oligonucleotides, to provide additional determination of the preselected relationship, wherein the last repeat of steps f through i provide final extender oligonucleotides; and
k. incubating the substrate with a plurality of readout oligonucleotides, each readout oligonucleotide comprising a detectable label bound to an oligonucleotide complementary to the bridging sequence on the final extender oligonucleotide, thereby detecting the extent of bound final extender oligonucleotides on the substrate; wherein
   i. each first weight oligonucleotide comprises two segments, one segment comprising a detector sequence complementary to a nucleic acid molecule species or sequence therein of interest, and the other segment comprising a bridging sequence complementary to a first blocking oligonucleotide or a first extender oligonucleotide;
   ii. each first blocking oligonucleotide comprises a predetermined affinity to the bridging sequence of the first weight oligonucleotide;
   iii. each first extender oligonucleotide comprises two segments, a detector sequence complementary to the bridging sequence of the first weight oligonucleotide, and a bridging sequence complementary to the detector sequence on a second weight oligonucleotide;
   iv. each second weight oligonucleotide comprises two segments, one segment comprising a detector sequence complementary to the bridging sequence of the first extender oligonucleotide, and the other segment comprising a bridging sequence complementary to a second blocking oligonucleotide or a second extender oligonucleotide;
   v. each second blocking oligonucleotide comprises a predetermined affinity to the bridging sequence of the second weight oligonucleotide; and
   vi. each second extender oligonucleotide comprises two segments, a detector sequence complementary to the bridging sequence of the second weight oligonucleotide, and a bridging sequence complementary to the detector sequence on a readout oligonucleotide or the bridging sequence of a further weight oligonucleotide; and wherein the extent of binding of the readout oligonucleotides to the substrate provides the at least one preselected relationship among the plurality of nucleic acid molecule species in the sample.

In some embodiments of any of the foregoing methods, the detector sequence and bridging sequence on an extender oligonucleotide are the same. In some embodiments of the foregoing methods, the blocking oligonucleotides are incubated with the substrate before the extender sequences are added. In some embodiments of the foregoing methods, the blocking oligonucleotides, if used, are incubated with the substrate, then unbound blocking oligonucleotides are removed before incubating the substrate with the extender sequences. In some embodiments thereof, the affinity of the blocking oligonucleotide for the bridging sequence is comparable to the affinity of the detector sequence on the extender oligonucleotide. In some embodiments thereof, the affinity of the blocking oligonucleotide for the bridging sequence is higher than the affinity of the detector sequence on the extender oligonucleotide. In some embodiments thereof, the affinity of the blocking oligonucleotide for the bridging sequence is at least 10-fold higher than the affinity of the detector sequence on the extender oligonucleotide.

In some embodiments of any of the foregoing methods, the blocking oligonucleotides and extender sequences are incubated with the substrate at the same time. In some embodiments of the foregoing method, the affinity of the blocking oligonucleotide for the bridging sequence is higher than the affinity of the detector sequence on the extender oligonucleotide. In some embodiments of the foregoing method, the affinity of the blocking oligonucleotide for the bridging sequence is at least 10-fold higher than the affinity of the detector sequence on the extender oligonucleotide.

In some embodiments of any of the foregoing methods, the preselected relationship is the amount of a nucleic acid molecule species of interest in the sample. In some embodiments of the foregoing methods, the preselected relationship is the relative amount of at least two nucleic acid molecule species of interest in the sample. In some embodiments of the foregoing methods, the preselected relationship is a relative amount of a panel of nucleic acid molecule species of interest in the sample.

In some embodiments of any of the foregoing methods, the extent of binding of the readout oligonucleotide reflects the amount of a nucleic acid species of interest in the sample. In some embodiments of the foregoing methods, the extent of binding of the readout oligonucleotide reflects the relative amounts of at least two nucleic acid species of interest in the sample. In some embodiments of the foregoing methods, the extent of binding of one or more readout oligonucleotides reflects the relative amounts of at least two nucleic acid species of interest in the sample. In some embodiments of the foregoing methods, the preselected relationship is a relative amount of a panel of nucleic acid molecule species of interest in the sample.

In some embodiments of any of the foregoing methods, one repeat of steps f through i are conducted. In some embodiments of the foregoing methods, two repeats of steps f through i are conducted.

In some embodiments of any of the foregoing methods, prior to incubating the substrate with a plurality of readout oligonucleotides, the substrate is incubated with a plurality of further weight oligonucleotides, wherein the detector sequences of the further weight oligonucleotides bind to any complementary nucleic acid molecule species bound to the substrate, and the readout oligonucleotides bind to the detector sequences of the further weight oligonucleotides.

In one embodiment, a method is provided for determining the presence of cancerous cells in a biopsy sample wherein a preselected relationship among levels of a plurality of nucleic acid molecules therein is diagnostic for cancer, comprising carrying out the method of any of the foregoing embodiments on nucleic acid molecules from the cancer cells bound to the substrate, and diagnosing the presence or absence of cancer therein.

In one embodiment, a method is provided for determining the type of cancer in a biopsy sample wherein a preselected relationships among levels of a plurality of nucleic acid molecules therein is diagnostic for a plurality of types of cancer, comprising carrying out the method of any of the foregoing embodiments on nucleic acid molecules from the cancer cells bound to the substrate, and diagnosing the type of cancer therein.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTIONS OF THE DRAWINGS

FIG. 1 depicts the general schematic of a multilayer perceptron (MLP).

FIG. 2 depicts the logic unit of the MLP.

FIG. 3 depicts the mathematical function of the rectified linear unit (ReLU).

FIG. 4 depicts the components of the nucleic acid-based MLP (NAMLP) disclosed herein.

FIGS. 5A-D is an illustration of nucleic acid based implementation of artificial neural network (NAMLP). FIG. 5A. A simple four-layer artificial neural network that demonstrates the use of nucleic acid computing throughout the figure. The network has two types of layers: Linear layers, i.e., matrix multiplication with non-negative weights (Layers #1 and #3, employing weight oligonucleotides), and non-linear layers (layers #2 and #4) that implement Rectifier Linear Units (ReLU) activation with non-positive biases (employing blocking oligonucleotides and extender oligonucleotides). The input to this layer was chosen to be [3.2.1] for demonstration purposes. In this example, the weight (Wi) and bias (Bi) matrices were chosen to simplify the drawing. In general, they are obtained using standard backpropagation optimization that maximizes the accuracy of label prediction. FIG. 5B. Matrix multiplication is achieved by designing “weight” nucleic acid oligos; each oligo links two nodes across two layers. The weight between every two nodes determines the number of “weight” oligos in the pool that maps between these two nodes. FIG. 5C. ReLU is achieved using high affinity competitive inhibitors (blocking oligonucleotides) that, if used, block a predefined number of binding sites on the weights oligos. Open sites on the “weight” oligos bind extender oligos, creating binding sites for the next layer. FIG. 5D. Demonstration of how the calculation needed for the exemplary network in panel (a) can be achieved using simple six steps of nucleic acid addition and washes. In this example, RNA molecules of interest are anchored to the surface using biotin/streptavidin to allow washes of excess oligos. The addition and washing steps create input-dependent nucleic acid “trees” with final leaves that encode the output of complex multistep calculations. At each step, the figure shows the status of the nucleic acid “tree” (left) and the corresponding status of the neural network (right). The nucleic acid computer performs calculations equivalent to modern machine learning algorithm (with minor constraints), thereby enabling the direct measurement of, for example, the position of cells in abstract latent space, optimized to contain information that is required for cell type label assignment.

### DETAILED DESCRIPTION

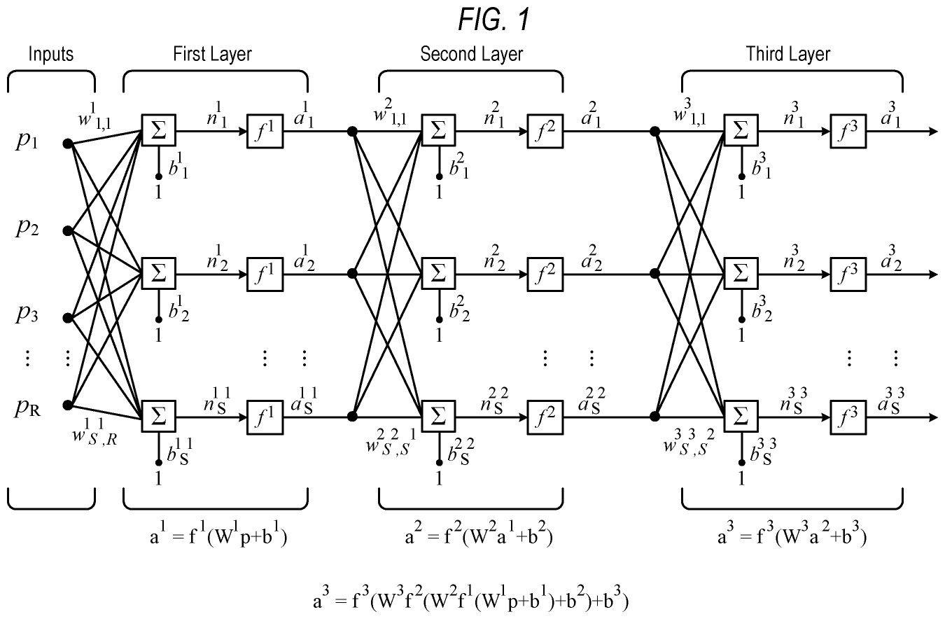

The disclosed methods provide for implementing a specific type of artificial neural network, a multilayer perceptron (MLP; FIG. 1), using a nucleic acid (such as DNA) to solve problems of regression and classification (nucleic acid-based MLP, or NAMLP). In some embodiments, the inputs of the molecular MLP are different amounts of multiple types of nucleic acid molecules. These nucleic acids could either be biological, e.g., DNA or RNA molecules in a cell, or synthetic encoding of information in a nucleic acid. In some embodiments, computations are based on a series of hybridization of complementary base-pairs of nucleic acids. In some embodiments, hidden layers of the MLP comprise oligonucleotides to carry out the activation function. In some embodiments, after the series of hybridization, the final output of the computation is read through final hybridization with nucleic acid oligomers (sometimes referred to herein as oligonucleotides or oligos, or oligonucleotide sequences or oligo sequences, or sequences, and syntactical variants thereof) that are conjugated to fluorophores, by way of non-limiting examples. In another embodiment, the final output is read by sequencing.

The method is generally based on the following steps:
1. Providing in-silico (e.g., on a standard computer) training to identify the required components based on labeled data. The training must follow specific requirements (described further below) to allow the optimization results to be implemented in molecules.
2. Design of a series of hybridizations based on the results of the in-silico training.
3. A series of simple pipetting steps of the design to a solution that has the input nucleic acid immobilized (e.g., to a surface, beads, fixed to other biological polymers in a cell, etc). The final steps use nucleic acids that are conjugated to fluorophores. In other embodiments the final step may comprise sequencing.
4. Fluorescent detection of the amount of each of the fluorophores using appropriate measurement (e.g., flow cytometer for cells, spectrometer for synthetic mixtures, plate reader, fluorescent microscopy. In another embodiment, sequencing of the nucleic acids in the final steps is performed.
5. Decoding of the readout based on parameters obtained during the optimization step in 1.

In some embodiments, Steps 1-2 need to occur once per problem type, whereas steps 3-5 are carried out each time such problem type needs to be solved. In some embodiments, further analysis or decoding of the readout may be performed on a computer, e.g., using additional calculation layers using traditional computer to extend the calculation performed by the nucleic acid-based MLP.

Non-limiting examples of the application of the NAMLP to a biological sample include cancer type screening, where the NAMLP reagents are determined that provide a readout of the specific type of cancer cells in a biological sample. Another example is use in cell type scanning to identify certain types of cells in a specimen. Another example is classification of tissue to be inflamed or non-inflamed. Different microbiomes could be classified based on predefined categories, for example, a microbiome that is helpful in weight reduction.

For example, where available data can correlate the presence and/or levels of nucleic acids in a cell as indicative of cancer, using the NAMLP disclosed herein, a method for determining the presence of cancerous cells in a biopsy sample is provided wherein a preselected relationship among levels of a plurality of nucleic acid molecules therein is diagnostic for cancer, comprising carrying out the NAMLP as described herein on nucleic acid molecules from the cancer cells bound to the substrate, and diagnosing the presence or absence of cancer therein.

In another example where certain types of cancer are determinable from a particular pattern of nucleic acid expression in a cell, a method fusing the NAMLP disclosed herein for determining the type of cancer in a biopsy sample is provided, wherein a preselected relationships among levels of a plurality of nucleic acid molecules therein is diagnostic for a plurality of types of cancer, comprising carrying out the NAMLP on nucleic acid molecules from the cancer cells bound to the substrate, and diagnosing the type of cancer therein. These and other applications of the NAMLP will be apparent.

Each of the components and steps in the molecular MLP (also referred to herein as a nucleic acid-based MLP, or NAMLP) are described further below. The following descriptions, and the ensuing examples, are merely illustrative of the way the nucleic acid computing is carried out and is not limiting as to variations therein that provide the desired outcome.

The core of the invention is the parallel between one specific form of an artificial neural network, a multilayer perceptron (MLP; see FIG. 1) having a specific type of activation function, and a specific protocol for the design and hybridization of DNA oligomers. The specific form of MLP is described below, followed by a description of its molecular implementation.

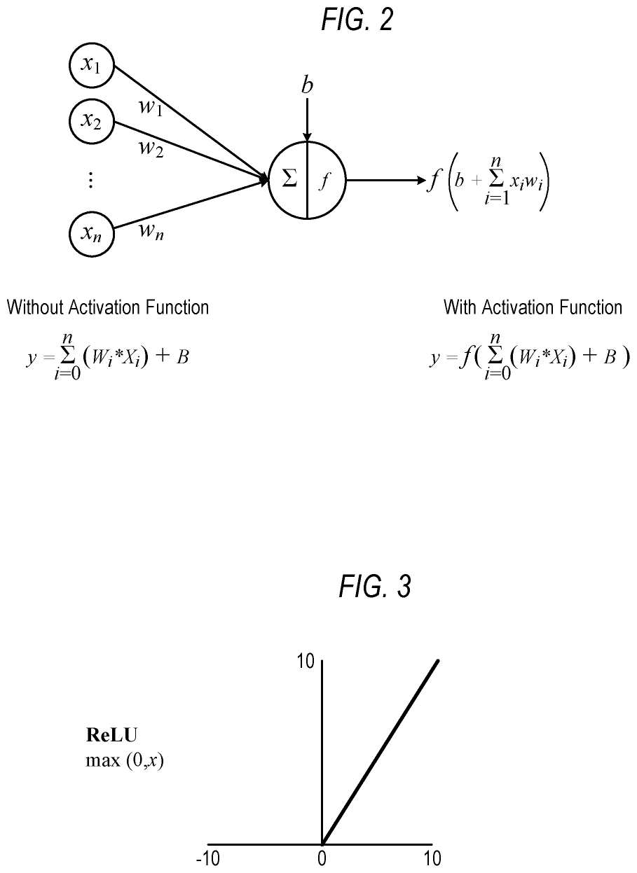

MLP design. The basic logic unit of a MLP is shown in FIG. 2. As disclosed herein, the NAMLP follows a similar design, utilizing input nucleic acid sequences and hybridizing further nucleic acids (for building layers or blocking them). As input, the NAMLP gets a vector Yº of strictly positive values with dimensionality D⁰. The multilayer perceptron has k internal hidden layers where Y^{k} is the k^{th} layer with D^{k} units (dimension of the vector Y^{k}). Two types of layers are possible, linear mapping implementing matrix multiplication and non-linear activation function in the form of Rectified Linear Unit (ReLU; FIG. 3):
1. a linear mapping between layer k−1 and k. This mapping is simply matrix multiplication with a weight matrix W^{k} between layer k−1 to layer k. It has a dimension D^{k}×D^{k-1}. Achieved in the NAMLP using weight oligonucleotides.
2. A nonlinear ReLU layer with strictly non-positive biases B^{k} such that Y^{k}=max(Y^{k-1}+B^{k},0). Achieved in the NAMLP using blocking oligonucleotides and extender oligonucleotides.

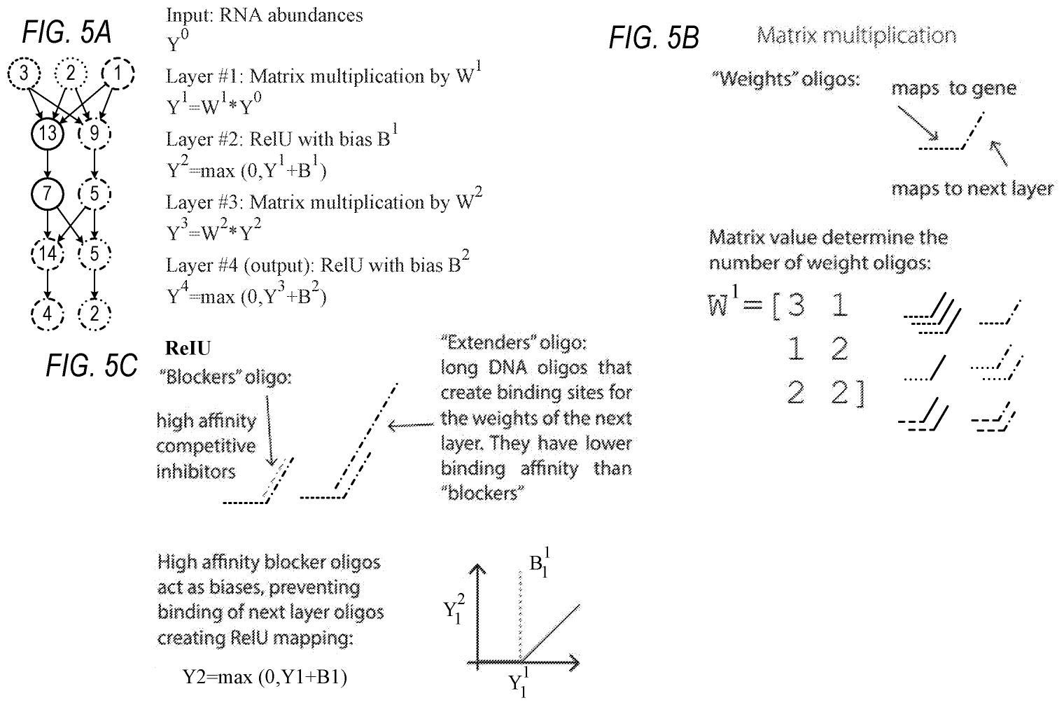

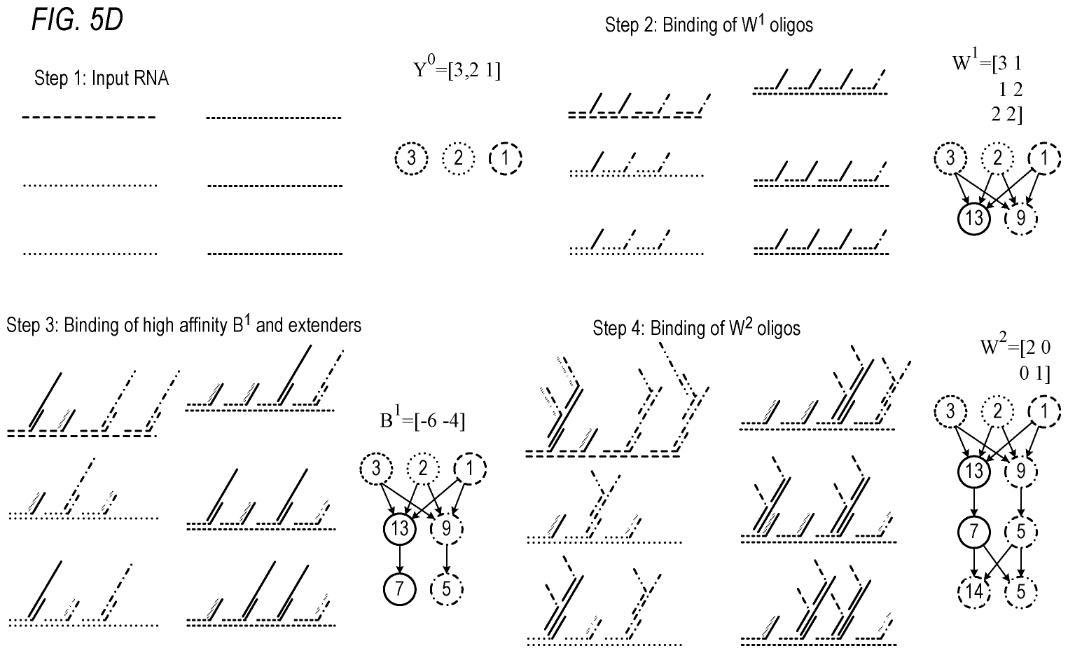

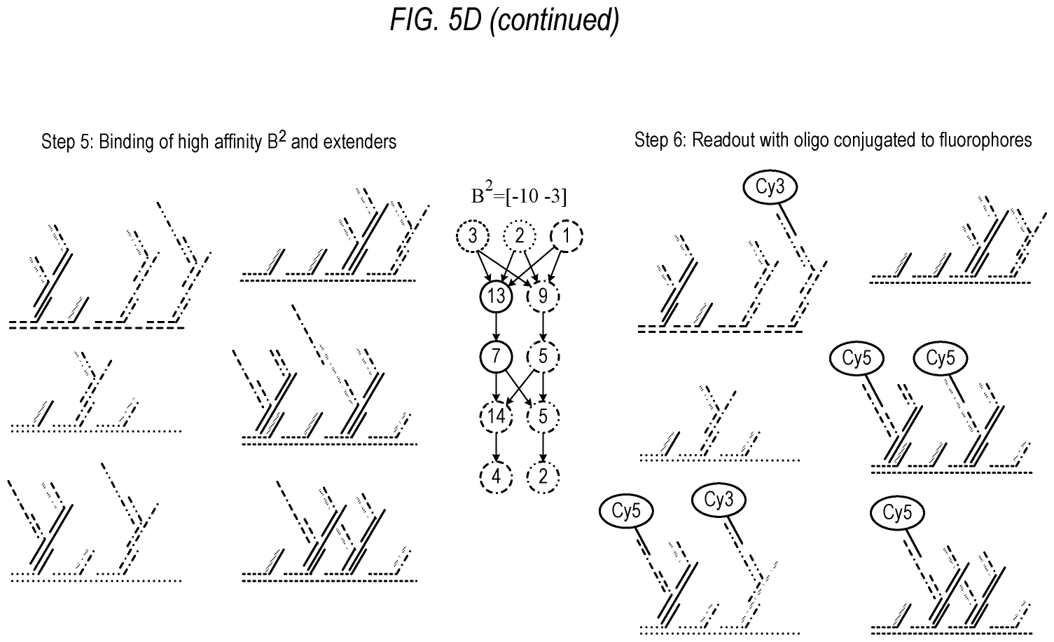

The two types of layers are typically used one after the other such that in vector notation the nonlinearity used is Y^{k+1}=max (0, Y^{k-1} W^{k}+B^{k}),0). In some embodiments, one or more further hidden layers comprising the linear mapping, the non-linear ReLU layer, or the combination of the linear mapping step and non-linear ReLU layer step, are used. FIG. 5A shows a simple NAMLP with two sets of the combination of the linear mapping and non-linear ReLU layer, comprising the hidden layers (corresponding oligonucleotide reagents may be referred to as first weight oligonucleotides, first blocking oligonucleotides and first extender oligonucleotides for the first two layers [linear then non-linear], followed by second weight oligonucleotides, second blocking oligonucleotides and second extender oligonucleotides for the next two layers [linear then non-linear]). However, the disclosure is not so limited and any additional number of linear and non-linear mapping layers may be included for further encoding the information. In certain embodiments, any one or more layers may comprise only linear mapping, and therefore not use any blocking oligonucleotides.

Additional layers. After the k+1 layer (kth hidden layer) the network can include additional number of layers that further decode the information contained in the k+1 layer. These additional layers can use any form of computation as they, in some embodiments, will not be implemented in DNA oligos and only calculated in silico.

Overall, the network is fully specified by its architecture, i.e. the number of layers that will be implemented in DNA oligos, their dimensionality, i.e. the number of hidden units in each layer, and the number of additional decoding layers, their dimensionality, and the choice of activation functions in these decoding layers. In one embodiment, the activation function is a ReLU. In another embodiment, the network could be constructed without an activation function in which case the overall NAMLP will implement a linear function.

Given a network specification, the network is trained based on labeled data. The labeled data need to include the inputs, i.e. concentration of nucleic acids (DNA or RNA) at the input layer and the final fully decoded information. In classification problems that will be a label, in regression problems that will be a continuous variable.

In this specific form of MLP, the parameters have additional constraints that are needed to allow the k+1 first layers of the network to be implemented in DNA oligos.
1. All weights parameters between linear layers are strictly non-negative (W^{k} for all k) whereas all RelU parameters (B^{k} for all k) are strictly non-positive.
2. Inputs are nonnegative (Y⁰>=0).
3. The values of W are discrete (this could be done by binning after training continuous W).
4. The W matrices are sparse. The key constraint is that the sum over a column in each of the matrices Wk is smaller than Nₛᵢₜₑₛ where Nₛᵢₜₑₛ is a tunable parameter that depends on the ability to synthesize DNA oligos. Typically Nₛᵢₜₑₛ will be ˜10. Nₛᵢₜₑₛ is determined by the length of the extender oligo (FIG. 5C).

The training will specify the values of network parameters, W^{k} and B^{k} for the first k hidden layers as well as additional parameters for the decoding layers. The values of W^{k}, and B^{k} will be used for the design of the molecular implementation of the first kth layer in the MLP.

### Implementation of the NAMLP

Input layer. In some embodiments, the input layer can either be a naturally occurring set of nucleic acids such as RNA and DNA in a given sample, or a synthetic encoding of information. The synthetic encoding is based on one or more DNA oligos that have hybridization targets, each target is of length N_{target} (N_{target}=20 is used for the examples below), but that value can be further optimized as DNA oligo synthesis technologies improve. For example, an 8×12 image with 256 grayscale levels can be converted to a mixture of 96 20mers, each with concentration 0-255 nM. The length of each of these oligos is Nₛᵢₜₑₛ*N_{target} bases. Each N_{target} in each of these oligos has to be unique and have good binding properties (GC content, melting temperature, secondary structure, etc.). In synthetic encoding, in one embodiment, the mixture could potentially be combined to create a single information-encoding long molecule. To allow washing, the input molecules need to be immobilized (e.g., bottom of a well, immobilized on beads, bound to cells, fixed to a slide, etc.). Such immobilization may be performed by any of a number of methods well known in the art. Thus, in one embodiment, a plurality of nucleic acid molecule species are bound to a substrate. In non-limiting embodiment, the substrate may be a bead, a glass slide, a polymer, a cell surface, a tissue surface, a biological specimen, a plastic surface, or a microtiter plate well.

Each hidden layer. Each hidden layer is represented by Dₖ^{k} unique oligos (weight oligos). Each of these oligos has two parts. The first is a unique sequence of size N_{target} that represents the specific unit i in hidden layer k (Yᵢ) and is complementary to the extender oligonucleotide sequences Ak and the other is a set of Nsite*Ntarget unique sequences that provide potential binding sites for the Wk+1 weight oligos. The value of Yk is just a vector of the counts of each of these oligos in solution.

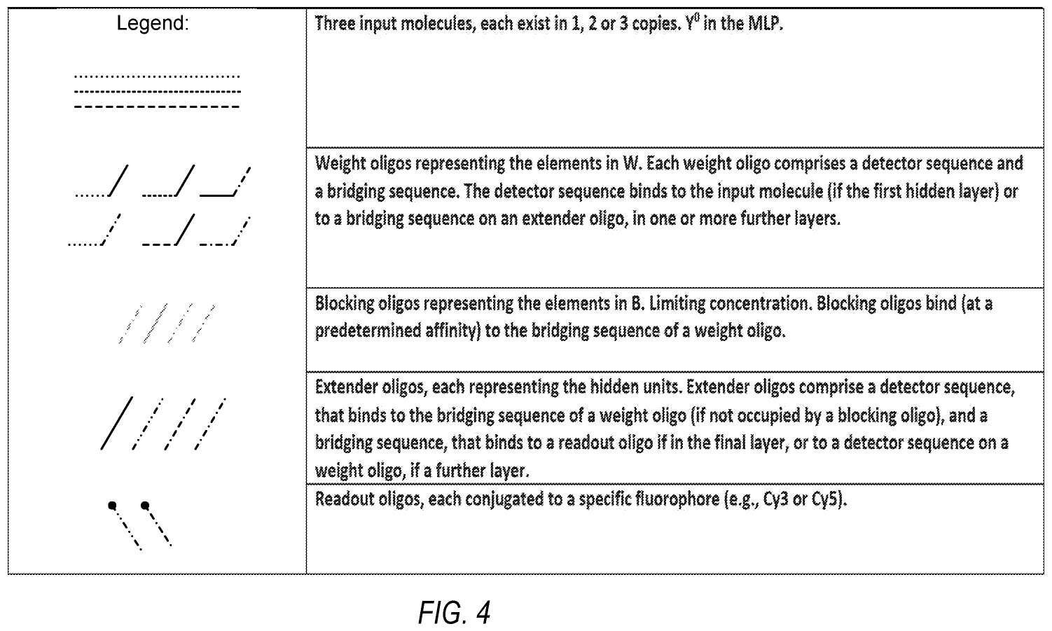

The weight matrices. The weight matrix is a set of DNA oligos each of length 2*N_{target}, i.e. 40mers in our examples. The size of the set is the sum of all the positive values in the matrix W^{k}. For a specific value W^{k}ᵢⱼ that maps the units Y^{k-1}ⱼ into Y^{k}ᵢ, the number of oligos needed is just the values of W^{k}ᵢⱼ where each of these 40mer has two halves (FIG. 4, FIG. 5b). The first 20mer (N_{target}) is complementary to one of the input molecule's binding sites 20mer in Y^{k-1}ⱼ, and the other is complementary to extender oligos that provide the next set of binding sites of the next layer.

In some embodiments, the weight oligonucleotide (weight oligo) comprises two segments: a detector sequence that is complementary to a nucleic acid molecule species or sequence therein of interest, or, in a NAMLP comprising more than one weight layer, is complementary to a bridging sequence on an extender oligonucleotide. The other segment of the weight oligo comprises a bridging sequence complementary to a blocking oligonucleotide (when used) or complementary to the detector sequence of a extender oligonucleotide.

Activation function (i.e. nonlinear transformation). We achieve the Y^{k}=max (0, Yₖ₋₁ W^{k}+B^{k}) activation function, if used, by two additional sets of DNA oligo for each layer. The set of blocking oligos is used to represent B^{k}. These blocking oligos have the same target site as extender oligos but with a much higher affinity for the binding sites (of length N_{target}) than the extender oligos. The relative affinity can be further optimized but has to be at least 10× higher affinity. In another embodiment, the affinity could be comparable and the blocker oligos will be added and washed prior to addition of extender oligos. In some embodiments the extender oligos and blocking oligos can be added simultaneously or in succession, without washing. The blocking oligos B^{k} will be added in at limiting concentration, and due to their much higher affinity or order of addition and thereby will “zero” out the first [B^{k}] sites effectively creating a threshold value. In some embodiments, no blocking oligonucleotides are added, thus providing a linear mapping and not limiting the binding sites of extender oligonucleotides.

In some embodiments, each blocking oligonucleotide comprises a predetermined affinity to the bridging sequence of the weight oligonucleotide to which it is complementary. Such predetermined affinity is established during the encoding calculations performed to establish the relationship as described herein. As noted herein, in some embodiments, encoding may indicate that no blocking oligonucleotides are added, thus not limiting the binding sites of extender oligonucleotides for any one or more layers of the NAMLP.

In some embodiments, each extender oligonucleotide comprises two segments, a detector sequence complementary to the bridging sequence of a weight oligonucleotide, and a bridging sequence complementary to either a readout oligonucleotide (where the extender oligo is the final extender oligo) or complementary to the detector sequence on a weight oligonucleotide (to comprise the next or further layer of the NAMLP). In certain embodiments, a final extender oligonucleotide may not be in the final layer of the NAMLP if any extender oligonucleotide at any layer of the NAMLP is not bound by any further weight oligonucleotide, as may be designed in the encoding of the network. The disclosure is thus not limiting as to the layers at which the readout oligonucleotides bind.

In some embodiments, the same detector or bridging segments, or readout oligo sequence, may be used in more than one oligonucleotide and/or in more than one layer. One of skill in the art will design, based on the disclosure herein, an efficient way of designing the components of the various oligonucleotides, the number of hidden layers, the binding affinity of the blocking oligonucleotides (if used), and other parameters, to provide the desired readout of the NAMLP based on the sample and desired calculation based thereon.

Pipetting scheme. The first layer is bound to a surface to allow washes. The oligos representing the hidden layers and weight matrix are added at high concentrations and washed at each step. Each pipetting step is followed by mixing to make sure that the solution is well mixed. The following molecular biology steps will be repeated for each of the kth hidden layers:
1. Pipette matrix W^{k} oligos (weight oligos)
2. Wash.
3. Pipette blockers B^{k}
4. Pipette in next layer oligos (extenders) Y^{k}
5. Wash

The final step is the addition of readout probes (pipette, mix, and wash excess). Readout probes are DNA oligos that are complementary to the sequences of layer Y^{k} conjugated with a fluorophore. The fluorescence of the sample is measured with standard tools (microscope, flow cytometer, spectrophotometer). In some embodiments, a wash step between the addition of blocker oligonucleotides and the addition of extender oligonucleotides may be provided, such that the affinity of the blocker oligonucleotides vs. the extender oligonucleotides can be comparable.

FIG. 4 shows the different oligos used in the design of the NAMLP in the non-limiting example described in FIGS. 5A-D.

FIG. 5A shows an example of a NAMLP with four hidden layers to depict how one specific instance of an NAMLP network can be implemented through a series of DNA hybridizations. In FIG. 5A, a simple four-layer artificial neural network that demonstrates the use of DNA computing throughout the figure. The network has two types of layers: Linear layers, i.e., matrix multiplication with non-negative weights (Layers #1 and #3), and non-linear layers (layers #2 and #4) that implement Rectifier Linear Units (ReLU) activation with non-positive biases. The input to this layer was chosen to be [3.2.1] for demonstration purposes. In this example, the weight (Wi) matrices and bias (Bi) matrices (comprising blocking oligos and extender oligos) were chosen to simplify the drawing. In general, they are obtained using standard backpropagation optimization that maximizes the accuracy of cell type classification. FIG. 5B shows that matrix multiplication is achieved by designing “weight” DNA oligos; each oligo links two nodes across two layers. The weight between every two nodes determines the number of “weight” oligos in the pool that maps between these two nodes. In FIG. 5C, ReLU is achieved using high affinity competitive inhibitors that block a predefined number of binding sites on the weights oligos. Open sites on the “weight” oligos bind extender oligos, creating binding sites for the next layer.

FIG. 5D is a demonstration of how the calculation needed for the network in FIG. 5A can be achieved using simple six steps of DNA addition and washes. RNA molecules are anchored to the surface using biotin/streptavidin (in one non-limiting embodiment) to allow washes of excess oligos. The addition and washing steps create input-dependent DNA “trees” with final “leaves” that encode the output of complex multistep calculations. The figure shows the status of the DNA tree (left) and the corresponding status of the neural network (right) at each step. The DNA computer performs calculations equivalent to modern machine learning algorithm (with minor constraints), thereby enabling the direct measurement of the position of cells in abstract latent space, optimized to contain information that is required for cell type label assignment. It should be noted that the final leaves may be at different levels of the branches of the tree.

Additional layers. As described herein, in its basic form, the NAMLP comprises two layers in addition to the sample and the readout layer: 1) weight oligos, 2) blocking/extender oligos (used together or in succession; in some embodiments, no blocking oligo is used). In other embodiments, the NAMLP comprises further layers of 1) weight oligos and 2) blocking/extender oligos (used together or in succession; in some embodiments, no blocking oligo is used). In some embodiments, a final weight oligo layer may be included. Thus, in some embodiments, the NAMLP layers comprising the weight oligos and the blocking/extender oligos (used together or in succession, or only extender oligos are used) are repeated once, thus providing a four layer NAMLP. In some embodiments, NAMLP layers comprising the weight oligos and the blocking/extender oligos (used together or in succession, or only extender oligos are used) are repeated twice, thus providing a six layer NAMLP. In some embodiments, further layers comprising the weight oligos and blocking/extender oligos (used together or in succession, or only extender oligos are used) are used. In some embodiments, such further layers are provided to further calculate the output of the desired analysis of the nucleic acid molecules in the sample. The last bound extender oligos without further bound weight oligos provide, at any level of the NAMLP, the sites for binding of the readout oligos.

For example, a NAMLP disclosed herein may comprise 1) first weight oligos and, 2) first blocking oligos and first extender oligos (together or in succession). In another example, a NAMLP as disclosed herein may comprise 1) first weight oligos, 2) first blocking oligos and first extender oligos (together or in succession), 3) second weight oligos and 4) second blocking and second extender oligos (together or in succession). In another example, a further layer of weight oligos, blocking and extender oligos may be used, such that the NAMPL comprises 1) first weight oligos, 2) first blocking oligos and first extender oligos (together or in succession), 3) second weight oligos 4) second blocking and second extender oligos (together or in succession), 5) third weight oligos and 6) third blocking and third extender oligos. As noted herein the last extender oligos in the NAMLP that are detected by the readout oligos may be referred to as final extender oligos. In certain embodiments, the final extender oligos may be on any of the layers of the NAMLP. In other embodiments, any of the foregoing examples may omit the first and/or second and/or third blocking oligonucleotides.

Problem Types. The NAMLP may be used to solve any number of problems that are implemented in nucleic acid molecules, wherein the NAMLP is used for at least one step in the computational analysis of data. The inputs of the NAMLP may be nucleic acids and the output comprises analyzing nucleic acid sequences; in another embodiment, the inputs of the NAMLP are derived from non-nucleic acid data. In another embodiment, the output of the NAMLP is the detection of fluorophores conjugated to oligonucleotides. In some embodiments, the output of the NAMLP, whether nucleic acid, fluorophore, or other readout, undergoes further computational analysis by another method such as in silico. In some embodiments, the problem to be solved is entirely NAMLP based. In some embodiments, the NAMLP is at the initiation of the analysis. In some embodiments the NAMLP is preceded and followed by non-NAMLP computational methods. In some embodiments, the NAMLP is the last step of the computational analysis. In other embodiments, the computational analysis comprises NAMLP.

As will be described in the examples below, one problem type is the high-level classification of cell types in a biological specimen, e.g., identification of cancer cell type in a solid or liquid biopsy specimen; and the high-level mapping of the location of such cell types within the specimen, where each cell type is distinguishable from each other cell type by the absence or presence of biological markers, or level of expression of such markers if present, typically involving dozens of biological markers levels of which are continuously varied among each different cell type, thus comprising a large data set from which high-level information can only be rapidly or readily discerned by computational analysis of the data. Where such biological markers are nucleic acids specific to each cell type, or by using reagents that convert each biological marker into a nucleic acid, the NAMLP disclosed herein can be used to reduce the plethora of data into a high-level map of cell types in a specimen.

Methods for designing the required oligonucleotides and steps to carry out the NAMLP may be performed in silico, where the input information includes the complexity of the biological system to be analyzed and the desired type of information to be read out from the NAMLP. Based on the number of different input nucleic acids to be analyzed and the output (e.g., binary [yes or no] to a ratio or more signals) the NAMLP is appropriately designed. The oligonucleotide reagents, their affinities, etc., are designed in silico then may be tested and refined by in vitro evaluation. The methods for reading out the results of the NAMLP are also determined, whether a direct readout or data needing further, e.g., in silico, analysis. Once the NAMLP is designed and the reagents available, specimens may be studied.

In another example of a problem type, the analysis of a genome to determine cell type origin (e.g., species), gender, the presence of patterns or clusters of genes of potential detriment, etc., can be analyzed using input DNA from a sample (amplified if required), NAMLP reagents and output reader, to indicate the desired information.

The following examples are intended to illustrate certain embodiments of the present invention, but do not exemplify the full scope of the invention.

### EXAMPLES

### Example 1. Cancer Diagnostic

For a cancer diagnostic application of the NAMLP, the type of cancer can be identified based on creating a set of reagents that identify specific types of cancer and produce a readout from applying the NAMLP to a cellular sample (from e.g., a biopsy) affixed to a substrate.
Step a: use public databases such as The Cancer Genome Atlas to collect RNAseq datasets of health/disease individuals.
Step b: Train an MLP with the proper constraints where output is binary (e.g., yes/no cancer).
Step c: Use NAMLP to classify patient RNA samples extracted from biopsies to diagnose if the patient has a tumor.

### Example 2. DNA-Based MLP to Optimize High-Level Cell Type Locations

Mapping the locations within a specimen of specific cell types can be achieved using the methods disclosed herein to carry out the dimensionality reduction process required to convert a massive amount of spatial location and identity data into a concise depiction of the locations of important cell types at a high level, eliminating noise and reducing the importance of rare events. Such identification is not based on a “1:1” correlation between the cell type and its location as would be determined by conventional cell staining or even more advanced methods using immunocytochemistry or in-situ hybridization, where the specific position of a cell in a specimen is based on a detectable property (e.g., antibody binding, nucleic acid hybridization) at a position; such methods for identifying locations of numerous cells types in a large specimen are tedious, time consuming and often unnecessary in order to yield the desired information. In contrast, the methods described herein provide a higher level cell type classification within the specimen based on a plurality of properties of each cell type (e.g., receptor expression, nucleic acid expression), employing reagents and labels that maximally differentiate among the cell types and readily provides high-level cell type location. The NAMLP described herein provides the dimensionality reduction feature to extract such information from a massive data set.

The following descriptions of the steps of the method are exemplary and non-limiting. Variations that achieve the same or similar outcomes are fully embraced herein.

Selecting Cell Types within the Specimen.

The cell types to be located within a specimen a guided by the information desired to be obtained by locating the positions of such cell types within the specimen. By way of example, the distribution of cancer cells in stroma from a solid tumor biopsy, or the distribution of astrocytes and neuronal cells in the hippocampus, may be diagnostic for cancer invasiveness or neurodegeneration, respectively. Moreover, mapping of cell types using the methods disclosed herein using a normal cellular sample, specimen, tissue or organ may provide information such as what comprises a normal (e.g., healthy) cell type distribution against which to compare pathological or suspected pathological specimens. Changes in cell type distributions over time may provide methods for determining chronological or biological age from a specimen. For example, numerous cell types have been identified in the brain, which have been classified into different categories and subcategories. While the skilled artisan may readily be cognizant of the types of cells in a particular biological sample of interest in localizing, such information on cell type makeup of organs and tissues in numerous animal species is available in the literature.

### Selecting Cell Type Molecular Markers.

The disclosure herein is based on identifying locations of cells relying on detectable expression of molecular markers on each of those cell types. Such markers may be unique to a particular cell type, or the same markers can be expressed in different amounts, absolutely or relative to one or more other markers, among a number of different cell types. As noted herein, the subsequent steps in which reagents are designed to optimally distinguish among cells types based on expression of such markers and may inform the selection of the markers to be used for the identification, such that steps (b) and (c) are interrelated, and the order they are carried out may be reversed or iterative. To prepare the specimen for dimensionality reduction using the NAMLP, the cell markers to be detected as the input layer of the NAMLP must be oligonucleotides; if oligonucleotides present normally in each cell type are not the markers to be used for the cell type analysis, reagents are provided that provide a unique oligonucleotide sequence at the location of each cell type, such as by use of an antibody-oligonucleotide or ligand-oligonucleotide conjugate, the antibody or ligand recognizing the cell marker. In one embodiment, the first dimensionality reduction step in this example is provided by the selection of the properties of the conjugate (binding affinity of the to the marker, for example; detectability of the oligonucleotide in subsequence steps in the NAMLP). As noted herein, in silico (e.g., computer) methods may be used to further process data generated by the NAMLP; similarly; other methods may be used to prepare the data set for the NAMLP analysis.

The molecular markers of the selected cell types from step (a) may be identified from the literature. For example, the identity and levels of expression of cell surface markers among the numerous types of brain cells is known from the literature. Identities of markers expressed on or by numerous cell types in tissues and organs of numerous animal species are an expanding part of the scientific literature.

In one embodiment, the molecular markers are nucleic acid polymers. In a preferred embodiment, the nucleic acid polymers are RNA. Such markers provide the input layer of the NAMLP. In an alternative embodiment, the molecular markers are protein, which may be any cell-surface protein, receptor, transcription factor, antibody, or a combination thereof. In such embodiments, as mentioned above, conjugates to provide oligonucleotides corresponding to cellular markers required for the cell type analysis is provided. As noted above, in providing a high level mapping by dimensionality reduction, the markers for which such conjugates are needed will be determined by the in silico encoding methods described below.

### Establishing a Relationship.

In silico methods are used to design the reagent set based on the above information, both the conjugates mentioned above, and the NAMLP components. This step identifies and implements a lower-dimensional representation of oligonucleotide distribution in a biological specimen, followed by additional statistical learning steps that assign labels to cells in the lower dimensional space. Similar to one popular dimensionality reduction scheme called principal components analysis (PCA) that is often used to create a representation of gene expression data using the first 20-50 components, wherein cell type classification is not occurring in the original gene expression space rather in a dimensionality reduced space that captures enough information to accurately classify cells into types, the NAMLP provides the cognate analysis. Other dimensionality reduction method applied to single-cell RNA expression data, including discernment projection non-negative matrix factorization (dPNMF) and recursive partitioning, are achieved by the NAMLP disclosed herein. As noted elsewhere, steps before and after use of the NAMLP may employ such other dimensionality reduction methods.

Furthermore, such establishing steps provided for a particular tissue or any other biological sample type may then be used for any other specimen of the same tissue or biological sample type, such that these steps need to be performed only once per sample type. In a non-limiting example, information for carrying out such steps may be stored and subsequently retrieved and used for processing additional specimens, including having the reagents described already prepared and ready for use, such that rapid processing and analysis of cell types locations in incoming tissues from a biopsy specimen or tumor resection, can be performed quickly for guiding drug therapy, further surgery, or both. Specialized reagents for detecting rare, abnormal, diseased or aberrant molecular marker expression may also be provided for diagnostic purposes.

Preparing reagents. Provided with these encoded machine-learned data, a sets of oligonucleotide reagents corresponding to the components in the hidden layers of the NAMLP are prepared. In addition, the conjugates of oligonucleotides to bind to the cellular markers, and the conjugates of oligonucleotides with detectable reagents, are prepared. The steps for successive reagent incubation, washing, etc., for as many layers as designed, are as described elsewhere herein. After the NAMLP step is conducted, the locations of the detectable oligonucleotides is performed.

Imaging the specimen. In one embodiment, the dyes used in the preparation of the binding reagents are imaged using hyperspectral imaging, wherein all dyes at a particular location within the specimen are imaged simultaneously and the quantitative information on each dye present at that location recorded. In an alternative embodiment, the dyes are imaged using a sequential wavelength-limited imaging, the sample washed, and reimaged using stepwise imaging methodology. In other words, the last step in the NAMLP wherein the dye conjugates specific for each particular oligonucleotide are imaged, in one non-limiting example, each such conjugate may be prepared with the same dye, and imaging conducted sequentially.

For hyperspectral imaging in two dimensions, in one embodiment a hyperspectral epi-fluorescence/confocal microscope can be used. For three-dimensional samples, in one embodiment, a hyperspectral light-sheet microscope may be used. Non-limiting examples include that described by Jahr et al., NATURE COMMUNICATIONS 2015; 6:7990; Lavagnino et al., B_{IOPHYSICAL} J 2016; 111:409-417; Xu et al., O_{PTICS} E_{XPRESS} 2017; 25 (25): 31159-31173).

Correlating image with cell type. To later decode the captured image data as described above and assign cell types, the NAMLP design provides the interpretation of the cell types at the locations within the specimen.

This method is able to map the abundance of ˜9,000 markers (e.g., RNA types) into 24 aggregate measurements such that the information on the label in each of these measurements is preserved.

Identify locations of specific cell types. The data on specific cell types and their locations obtained in step (g) are provided as a map or other data format to identify cell type locations within the specimen.

In one embodiment, the molecular markers are nucleic acid polymers. In a preferred embodiment, the nucleic acid polymers are RNA. In an alternative embodiment, the molecular markers are protein, which may be any of a secreted protein, cell-surface protein, receptor, transcription factor, antibody, or a combination thereof. As noted above, for non-nucleic acid markers, a conjugate of an oligonucleotide and a ligand to the marker (e.g., an antibody) is needed.

In one embodiment, the first three steps are performed for a particular type of biological specimen wherein the specimen comprises a plurality of known cell types (e.g., known from the literature) and among the known cell types within the specimen from which the plurality are selected for locating, the known molecular markers of each cell type is obtained from the literature.

In one embodiment, the locations of the cell types within the specimen are used diagnostically to identify, for example, a disease state or the potential for a diseases state to develop based upon the locations of particular cell types within the specimen.

What is claimed is:

1. A method for identifying at least one preselected relationship among a plurality of nucleic acid molecule species in a sample, the method comprising the steps of:
a. binding the plurality of nucleic acid molecule species to a substrate;
b. incubating the substrate with a plurality of first weight oligonucleotides, wherein the detector sequences of the first weight oligonucleotides bind to any nucleic acid molecule species complementary thereto bound to the substrate;
c. removing unbound first weight oligonucleotides;
d. incubating the substrate with first extender oligonucleotides and optionally first blocking oligonucleotides, wherein detector sequences on the first extender oligonucleotides or the first blocking oligonucleotides, if used, bind to sites complementary thereto on the bridging sequences of the first weight oligonucleotides, the extent of the binding of the first extender nucleotides based on the predetermined affinity of the first blocking oligonucleotides, if used, and of the detector sequences of the first extender oligonucleotides to the bridging sequences of the first weight oligonucleotides;
e. removing unbound first extender oligonucleotides and first blocking oligonucleotides if used, and;
f. wherein the bound first extender oligonucleotides are final extender oligonucleotides, or wherein optionally one or more repeats of steps b through e are conducted with further weight oligonucleotides, optional further blocking oligonucleotides and further extender oligonucleotides, to provide additional determination of the preselected relationship, and wherein the last repeat of steps b through e provide final extender oligonucleotides; and
g. incubating the substrate with a plurality of readout oligonucleotides, each readout oligonucleotide comprising a detectable label bound to an oligonucleotide complementary to the bridging sequence on the final extender oligonucleotide, thereby detecting the extent of bound final extender oligonucleotides on the substrate; wherein
i. each first weight oligonucleotide comprises two segments, one segment comprising a detector sequence complementary to a nucleic acid molecule species or sequence therein of interest, and the other segment comprising a bridging sequence complementary to a first blocking oligonucleotide or to the detector sequence of a first extender oligonucleotide;
ii. each first blocking oligonucleotide comprises a predetermined affinity to the bridging sequence of the first weight oligonucleotide; and
iii. each first extender oligonucleotide comprises two segments, a detector sequence complementary to the bridging sequence of the first weight oligonucleotide, and a bridging sequence complementary to the detector sequence on a further weight oligonucleotide or readout oligonucleotide; and
wherein the extent of binding of the readout oligonucleotides to the substrate provides the at least one preselected relationship among the plurality of nucleic acid molecule species in the sample.

2. A method for identifying at least one preselected relationship among a plurality of nucleic acid molecule species in a sample, the method comprising the steps of:
a. binding the plurality of nucleic acid molecule species to a substrate;
b. incubating the substrate with a plurality of first weight oligonucleotides, wherein the detector sequences of the first weight oligonucleotides bind to any nucleic acid molecule species complementary thereto bound to the substrate;
c. removing unbound first weight oligonucleotides;
d. incubating the substrate with first extender oligonucleotides and optionally first blocking oligonucleotides, wherein detector sequences of the first extender oligonucleotides and the first blocking oligonucleotides, if used, bind to sites complementary thereto on the bridging sequences of the first weight oligonucleotides, the extent of the binding of the extender nucleotides based on the predetermined affinity of the first blocking oligonucleotides, if used, and of the first extender nucleotides to the bridging sequences of the first weight oligonucleotides;
e. removing unbound first extender oligonucleotides and first blocking oligonucleotides if used;
f. incubating the substrate with a plurality of second weight oligonucleotides, wherein the detector sequences of the second weight oligonucleotide bind to complementary bridging sequences on the first extender oligonucleotides;
g. removing unbound second weight oligonucleotides;
h. incubating the substrate with second extender oligonucleotides and optionally second blocking oligonucleotides, wherein the detector sequences of the second extender oligonucleotides and the second blocking oligonucleotides, if used, bind to sites complementary thereto on the bridging sequences of the second weight oligonucleotides, the extent of the binding of the second extender nucleotides based on the predetermined affinity of the second blocking oligonucleotides, if used, and on the second extender nucleotides to the bridging sequences of the second weight oligonucleotides;
i. removing unbound second extender oligonucleotides and second blocking oligonucleotides if used;
j, wherein the bound second extender oligonucleotides are final extender oligonucleotides, or wherein optionally one or more repeats of steps f through i are conducted with further weight oligonucleotides, optionally further blocking oligonucleotides and further extender oligonucleotides, to provide additional determination of the preselected relationship, wherein the last repeat of steps f through i provide final extender oligonucleotides; and
k. incubating the substrate with a plurality of readout oligonucleotides, each readout oligonucleotide comprising a detectable label bound to an oligonucleotide complementary to the bridging sequence on the final extender oligonucleotide, thereby detecting the extent of bound final extender oligonucleotides on the substrate; wherein
i. each first weight oligonucleotide comprises two segments, one segment comprising a detector sequence complementary to a nucleic acid molecule species or sequence therein of interest, and the other segment comprising a bridging sequence complementary to a first blocking oligonucleotide or a first extender oligonucleotide;
ii. each first blocking oligonucleotide comprises a predetermined affinity to the bridging sequence of the first weight oligonucleotide;
iii. each first extender oligonucleotide comprises two segments, a detector sequence complementary to the bridging sequence of the first weight oligonucleotide, and a bridging sequence complementary to the detector sequence on a second weight oligonucleotide;
iv. each second weight oligonucleotide comprises two segments, one segment comprising a detector sequence complementary to the bridging sequence of the first extender oligonucleotide, and the other segment comprising a bridging sequence complementary to a second blocking oligonucleotide or a second extender oligonucleotide;
V. each second blocking oligonucleotide comprises a predetermined affinity to the bridging sequence of the second weight oligonucleotide; and
vi. each second extender oligonucleotide comprises two segments, a detector sequence complementary to the bridging sequence of the second weight oligonucleotide, and a bridging sequence complementary to the detector sequence on a readout oligonucleotide or the bridging sequence of a further weight oligonucleotide; and
wherein the extent of binding of the readout oligonucleotides to the substrate provides the at least one preselected relationship among the plurality of nucleic acid molecule species in the sample.

3. The method of claim 1 or 2 wherein the detector sequence and bridging sequence on an extender oligonucleotide are the same.

4. The method of claim 1 or 2 wherein the blocking oligonucleotides are incubated with the substrate before the extender sequences are added.

5. The method of claim 4 wherein the blocking oligonucleotides are incubated with the substrate, then unbound blocking oligonucleotides are removed before incubating the substrate with the extender sequences.

6. The method of claim 4 or 5, wherein the affinity of the blocking oligonucleotide for the bridging sequence is comparable to the affinity of the detector sequence on the extender oligonucleotide.

7. The method of claim 1 or 2, wherein the blocking oligonucleotides and extender sequences are incubated with the substrate at the same time.

8. The method of any one of claim 4, 5 or 7, wherein the affinity of the blocking oligonucleotide for the bridging sequence is higher than the affinity of the detector sequence on the extender oligonucleotide.

9. The method of claim 8, wherein the affinity of the blocking oligonucleotide for the bridging sequence is at least 10-fold higher than the affinity of the detector sequence on the extender oligonucleotide.

10. The method of claim 1 or 2 wherein the preselected relationship is the amount of a nucleic acid molecule species of interest in the sample.

11. The method of claim 1 or 2 wherein the preselected relationship is the relative amount of at least two nucleic acid molecule species of interest in the sample.

12. The method of claim 1 or 2 wherein the preselected relationship is a relative amounts of a panel of nucleic acid molecule species of interest in the sample.

13. The method of claim 1 or 2 wherein the extent of binding of the readout oligonucleotide reflects the amount of a nucleic acid species of interest in the sample.

14. The method of claim 1 or 2 wherein the extent of binding of the readout oligonucleotide reflects the relative amounts of at least two nucleic acid species of interest in the sample.

15. The method of claim 1 or 2 wherein the extent of binding of one or more readout oligonucleotides reflects the relative amounts of at least two nucleic acid species of interest in the sample.

16. The method of any one of claims 2-15, wherein one repeat of steps f through i are conducted.

17. The method of claim 2-15, wherein two repeats of steps f through i are conducted.

18. The method of any one of claims 1-17, wherein prior to incubating the substrate with a plurality of readout oligonucleotides, the substrate is incubated with a plurality of further weight oligonucleotides, wherein the detector sequences of the further weight oligonucleotides bind to any complementary nucleic acid molecule species bound to the substrate, and the readout oligonucleotides bind to the detector sequences of the further weight oligonucleotides.

19. A method for determining the presence of cancerous cells in a biopsy sample wherein a preselected relationship among levels of a plurality of nucleic acid molecules therein is diagnostic for cancer, comprising carrying out the method of any one of claims 1-18 on nucleic acid molecules from the cancer cells bound to the substrate, and diagnosing the presence or absence of cancer therein.

20. A method for determining the type of cancer in a biopsy sample wherein a preselected relationships among levels of a plurality of nucleic acid molecules therein is diagnostic for a plurality of types of cancer, comprising carrying out the method of any one of claims 1-18 on nucleic acid molecules from the cancer cells bound to the substrate, and diagnosing the type of cancer therein.
